(19) United States
(12) Patent Application Publication
(10) Pub. No.: US 2018/0105795 A1
(43) Pub. Date: Apr. 19, 2018

(54) METHOD FOR PREPARATION OF EXTRACELLULAR MATRIX-MODIFIED DECELLULARIZED NERVE SCAFFOLD AND USE THEREOF

(71) Applicant: SUN YAT-SEN UNIVERSITY, Guangzhou (CN)

(72) Inventors: Yuan-shan Zeng, Guangzhou (CN); Ge Li, Guangzhou (CN); Jia-hui Sun, Guangzhou (CN)

(21) Appl. No.: 15/730,770

(22) Filed: Oct. 12, 2017

(30) Foreign Application Priority Data
Oct. 13, 2016 (CN) 201610905475.4

(51) Int. Cl. C12N 5/079; A61L 27/36; A61L 27/38; A61K 35/12

(52) U.S. Cl. CPC C12N 5/0618; A61L 27/3633; C12N 2533/90; A61K 35/12; A61L 27/3839

(57) ABSTRACT

An extracellular matrix-modified decellularized nerve scaffold and use thereof are provided. The extracellular matrix-modified decellularized nerve scaffold is prepared from a natural porcine optic nerve. The scaffold has a plurality of longitudinal channels and a plurality of transversal foramina intercommunicated with the longitudinal channels, which have relatively uniform diameters and relatively even distributions in the scaffold. The extracellular matrix-modified decellularized optic nerve scaffold of the present invention changes the poor microenvironment of existing decellularized material that lacks cell growth factors and nutrients, supports seeded cells to form neural networks in vitro or in vivo, and enables the connection of ascending nerve fibers or descending nerve fibers of the injured spinal cord to their target cells after transplantation.

### CROSS-REFERENCE TO RELATED APPLICATIONS

The present application claims the benefit of Chinese Patent Application No. 201610905475.4 filed on Oct. 13, 2016, the contents of which are hereby incorporated by reference.

### FIELD OF THE INVENTION

The present invention relates to an extracellular matrix-modified tissue engineering scaffold for repairing an injured spinal cord, and more particularly to an extracellular matrix-modified decellularized optic nerve scaffold having a plurality of longitudinal channels with relatively uniform diameters and relatively even distributions and a plurality of transversal foramina with relatively uniform diameters and relatively even distributions. The extracellular matrix-modified decellularized optic nerve scaffold of the present invention changes the poor microenvironment of existing decellularized material that lacks cell growth factors and nutrients, supports seeded cells to form neural networks in vitro or in vivo, and enables the connection of ascending nerve fibers or descending nerve fibers of the injured spinal cord to their target cells after transplantation.

### BACKGROUND OF THE INVENTION

In the repair of spinal cord injury, a transplanted tissue engineering scaffold bridges the injured area of the spinal cord and provides supports for the migration and growth of cells and nerve fibers, which is conducive to functional repair of the injured spinal cord. Therefore, the tissue engineering scaffolds have been widely used as a tool for researches on spinal cord injury. At present, an ideal solution for the repair of spinal cord injury is the implantation of an artificial biological material based on the injury mechanism, using biodegradable polymers to support tissues and carry cells and sustained-release drugs, etc. When it comes to biological materials and scaffold design, following factors should be taken into consideration: i) biocompatibility; ii) cable-like structure, that is beneficial to tissue orientation and provides porous guided structure; iii) cytocompatibility, that is essential for cell attachment, migration and axon growth; iv) physical properties (elasticity, strength, and toughness), that should be similar to host tissue; and v) nontoxicity of biodegradable products.

So far, existing scaffolds used in repairing spinal cord injury only provide growth environment for nerve fibers, the nerve fibers are not able to grow orderly or orientedly, as a result the ascending or descending nerve fibers cannot be connected to their target cells accurately. Therefore the existing scaffolds still lack improved therapeutic effects. Though polymer scaffolds may be synthesized with longitudinal channels to facilitate the alignment of nerve fibers, the accumulation of acid derived from the degradation of poly lactic-co-glycolic acid (PLGA) and other compounds adversely affects the injury repair. Hence it is important to find a natural material with no such adverse effects and prepare a scaffold with longitudinal channels to mediate the attachment, growth and elongation of nerve fibers therein. The longitudinal channels should not be isolated with each other, but should be intercommunicated with transversal foramina which enable contacts and information transmissions among nerve fibers. The diameter of the longitudinal channels or the transversal foramina for receiving a bundle of neuronal axons is required to range from tens of microns to a few hundred micrometers, but it is difficult to prepare tissue engineering scaffolds with uniform channels and foramina in small size as such at present. Therefore, the inventors of the present invention tried to develop applicable nerve scaffolds from natural biological tissues.

Intercellular spaces normally are filled with extracellular matrix, which plays a role in supporting, protecting and nourishing cells with no significant immunogenicity in general. In fields of tissue repairing, except for spinal cord injury repairing, the use of extracellular matrix to promote tissue repair has been reported to show great potentials in blood vessels, skins, bones, cartilages, livers, lungs, hearts, peripheral nerves and other organs. However, because the spinal cord tissue is soft in structure and complex in cell types, no satisfactory therapeutic effect has been achieved in the field of spinal cord injury repair using decellularized spinal cord scaffolds. Studies have shown that decellularized central nerve scaffolds could improve proliferation, migration and differentiation of PC12 cell line (Volpato F Z, Migliaresi C, Hutmacher D W, et al. Using extracellular matrix for regenerative medicine in the spinal cord. Biomaterials, 2013, 34(21):4945-55.) and also promote angiogenesis of chick embro chorioallantoic membranes (Ribatti D, Conconi M T, Nico B, et al. Angiogenic response induced by acellular brain scaffolds grafted onto the chick embryo chorioallantoic membrane. Brain Research, 2003. 989(1): p. 9-15.) in vitro, and hardly generated immunogenicity.

A previous research tested a variety of acellular extracellular matrix derived from central nervous system (CNS) tissues such as optic nerve, spinal cord, and brain (Crapo, P. M., Medberry C J, Reing J E, et al., Biologic scaffolds composed of central nervous system extracellular matrix. Biomaterials, 2012. 33(13): p. 3539-3547.). Few cell residues were found but neurosupportive proteins were retained in the extracellular matrix and the extracellular matrix were compatible with PC12 cells. However, the acellular extracellular matrix is wizened after lyophilization, in addition, longitudinal channels in optic nerve is collapsed and cannot be maintained. The optic nerve, also known as specialized cranial nerve II, has partitions divided by cerebral leptomeninx, nerve bundles are relatively evenly distributed in the optic nerve. As shown in a transversal cross-sectional view of the optic nerve, the diameter of each nerve bundle is about 100 μm. This is different from peripheral nerve, for example, nerve bundles of sciatic nerve are larger in diameter than that of optic nerve and the diameter are varied greatly among the nerve bundles of sciatic nerve. Therefore, the transplantation of decellularized optic nerve scaffolds, which is structurally similar to spinal cord white matter and beneficial for the formation, growth and extension of nerve bundle, has obvious advantages in repairing spinal cord injury.

Neural stem cells (NSCs) are a group of cells in nervous system that are undifferentiated, self-proliferative, self-renewable and potential to differentiate into neurons and glial cells. NSCs were first confirmed in subventricular zone of mouse in 1989 and isolated from striatum and ependymal layer of mouse in 1992 for the first time. The main function of NSCs is to serve as reserve cells for nerve tissue, replacing normal dead cells or participating in the repair of nerve tissue injury. NSCs can be transplanted for treatment of traumatic spinal cord injury based on the characteristics of self-renewal ability and differentiation potential of NSCs. Park et al. transplanted scaffolds made of PLGA and polylysine together with NSCs to mouse with brain injury and found that their motor function recovered and that the scaffolds itself could also reduce the formation of glial scars (Park, K. I., Y. D. Teng Y D and E. Y. Snyder E Y, et al. The injured brain interacts reciprocally with neural stem cells supported by scaffolds to reconstitute lost tissue. Nature Biotechnology, 2002. 20(11): p. 1111.). Then, a method of NSCs and Schwann cells (SCs) combined transplantation also achieved a good therapeutic effect. Results showed that this method promoted the neuronal axon myelination, providing favorable growth microenvironment for axonal formation; NSCs transplanted into the injured spinal cord could differentiate into neurons and glial cells, reduce the secondary injury of the spinal cord, protect the damaged neurons and promote the recovery of limb motor function (Guo, J. S., Zeng Y S, Li H B, et al., Cotransplant of neural stem cells and NT-3 gene modified Schwann cells promote the recovery of transected spinal cord injury. Spinal Cord, 2007. 45(1): p. 15; Zhang, X. B., Zeng Y S, Zhang W, et al., Synergistic effect of schwann cells and retinoic acid on differentiation and synaptogenesis of hippocampal neural stem cells in vitro. Biomedical and Environmental Sciences, 2006, 19(3): p. 219-224.).

At present, extracellular matrix-modified porous decellularized scaffolds derived from natural materials have not been reported yet. This inspired the inventors of the present invention to prepare an extracellular matrix-modified decellularized porcine optic nerve scaffold having a plurality of channels and foramina with relatively uniform diameters and relatively even distributions, which is promising for the formation of neural network-like structure in vitro and in vivo. Seed cells may be seeded into the bioactive extracellular matrix-modified decellularized optic nerve scaffold of the present invention, which is transplanted to the injury gap of ascending nerve fibers or descending nerve fibers so as to ultimately repair the spinal cord injury. Therefore, the object of the present invention is to overcome the existing shortcomings in the treatment of spinal cord injury in clinical practices by forming a neural network-like structure in the injury site using the extracellular matrix-modified decellularized optic nerve scaffold of the present invention.

### SUMMARY OF THE INVENTION

The present invention aims at providing an extracellular matrix-modified decellularized optic nerve scaffold having a plurality of longitudinal channels and a plurality of transversal foramina intercommunicated with the longitudinal channels. The scaffold of the present invention not only promotes seeded cells to form a neural network for information transmission, but also connects ascending or descending nerve fibers to their target cells in spinal cord injury sites after transplantation to play a role as an effective neuronal relay.

For the realization of the above-mentioned aim, the present invention provides an extracellular matrix-modified decellularized nerve scaffold, wherein the scaffold is derived from a natural porcine optic nerve, the scaffold has a plurality of longitudinal channels and a plurality of transversal foramina intercommunicated with the longitudinal channels, and the scaffold is loaded with extracellular matrix.

Preferably, the scaffold is prepared by decellularization, the plurality of longitudinal channels and the plurality of transversal foramina have relatively uniform diameters and relatively even distributions in the scaffold.

Preferably, the extracellular matrix is a cell growth factor and/or a cell nutritional factor; wherein the cell growth factor is selected from a group comprising neurotrophic factor-3 (NT-3), ciliary neurotrophic factor (CNTF), glial cell line-derived neurotrophic factor (GDNF), brain-derived neurotrophic factor (BDNF) and mixtures thereof; and the cell nutritional factor is selected from a group comprising fibroblast growth factor (FGF), insulin-like growth factor (IGF), or transforming growth factor (TGF) and mixtures thereof.

Preferably, the scaffold further comprises a cell secreting extracellular matrix, wherein the cell secreting extracellular matrix is selected from a group comprising Schwann cell and mesenchymal stem cell.

Preferably, the scaffold is seeded with seed cells to form a neural network-like structure which is able to be transplanted to an injured spinal cord and thus repair the injured spinal cord.

In the present invention, the decellularized porcine optic nerve scaffold is modified by extracellular matrix, then NSCs or dorsal root ganglion (DRG) was seeded into the modified scaffold, the nerve fibers grow along the channels and fromina in the scaffold to form a neural network-like structure which is able to be transplanted to an injured spinal cord and thus repair the injured spinal cord.

The beneficial effects of the scaffold of the present invention are that: after the transplantation of the extracellular matrix-modified scaffold with longitudinal channels and transversal foramina to the injured spinal cord, the neuron relay structure that has been formed in vitro facilitates the connection of ascending or descending nerve fibers to their target cells accurately in damage areas via the longitudinal channels, on this basis the transversal foramina serves as an additional connection pathway which builds cross-talks between the newly formed nerve fibers.

### BRIEF DESCRIPTION OF DRAWINGS

FIG. 1 is a schematic view of an extracellular matrix-modified decellularized porcine optic nerve scaffold implanted with seeded cells, wherein reference numerals and symbols show:
a: extracellular matrix-modified decellularized porcine optic nerve scaffold
1: longitudinal channels formed in scaffold
2: neuron
2-1: neuronal somata
2-2: nerve fibers

FIG. 2 shows an SEM (scanning electron microscope) picture of a decellularized porcine optic nerve scaffold, wherein arrows indicate longitudinal channels of the decellularized porcine optic nerve scaffold.

FIG. 3 shows an SEM (scanning electron microscope) picture of a decellularized porcine optic nerve scaffold, wherein arrows indicate transversal foramina of the decellularized porcine optic nerve scaffold.

### DETAILED DESCRIPTION OF ILLUSTRATED EMBODIMENTS

Embodiments of the present invention are further explained clearly as follows in conjunction with figures.

### Experimental Materials

Instruments: Benchtop (Suzhou Purification Electronic Equipment Factory, China); low-speed multi-tube auto-balancing centrifuge (Hunan Xiangyi Laboratory Instrument Development Co., Ltd., China); 5% CO₂ incubator (Queue, USA); inverted phase contrast microscope (Olympus, Japan); fluorescence microscope (Leica, Germany); scanning electron microscope (Philips, Netherlands); low temperature oven (Shanghai Yuejin Medical Equipment Factory, China); high temperature oven (Shanghai Yuejin Medical Equipment Factory, China); autoclave (Jiangyin Riverside Medical Equipment Factory, China); cryostat microtome (Shandon; UK); ultra-pure water purifier (Molsheim; France); shaker (Guangzhou Zhengyi Technology Co., Ltd., China); lyophilizer (Labconco, American).

Reagents: D-Hank's equilibrium liquid (self-prepared); 0.01 mol/L PBS (Zhongshan Jinqiao Biotech, China); Hoechst33342 (Sigma); goat serum (Zhonshan Jinqiao Biotech, China); sodium deoxycholate (Sigma); Triton X-100 (Shanghai Shenggong, China); β-NGF (Peprotech); B27 (GIBCO); Matrigel (GIBCO), DMEM/F12 (Hyclone); Neurobasal (GIBCO).

### Experimental Methods

1. Preparation of Extracellular Matrix-Modified Decellularized Optic Nerve Scaffolds

Porcine optic nerves were supplied by the Experimental Animal Center of Sun Yat-sen University. Tissue was placed in an ice box, separated from all non-optic nerve connective tissues and membranes and then frozen at −20° C. for storage.

Surface adipose tissue and some dura mater cerebralis were removed, and the porcine optic nerves were cut into 5 mm segments. The optic nerve segments were bathed and agitated in distilled water for 6 h at 60 rpm, bathed and agitated in 30 mL/L aqueous Triton X-100 solution for at 12 h at 60 rpm, rinsed with distilled water three times, bathed and agitated in 40 g/L (4%) aqueous sodium deoxycholate solution for 24 h at 60 rpm, rinsed with distilled water three times, repeatedly treated with said Triton X-100-distilled water-sodium deoxycholate-distilled water process two times, stored in PBS, fixed in 4% paraformaldehyde for 24 h, dehydrated in gradient sucrose solution for 24 h, bathed in 75% alcohol for 15 min, rinsed with D-Hank's solution three times with 10 min each time. The obtained sterilized decellurarized optic nerve materials were placed into vials that had been autoclaved sterilization-treated, and lyophilized in a lyophilizer for 12 h. Scanning electron microscopy was carried out to ascertain the longitudinal channels and transversal foramina of decellularized porcine optic nerve scaffold. Scaffolds were firstly washed 3 times with PBS, fixed in 2.5% glutaraldehyde for 90 min, dehydrated with a series of graded ethanol, and then freeze dried for 24 hours. The dried samples were coated with gold and examined under a scanning electron microscope (Philips XL30 FEG). The lyophilized sterilized decellurarized optic nerve scaffolds were immersed in extracellular matrix, mixed for 3 h, cross-linked and then stored dry until use.

In one embodiment, the extracellular matrix is a cell growth factor and/or a cell nutritional factor; wherein the cell growth factor is selected from a group comprising neurotrophic factor-3 (NT-3), ciliary neurotrophic factor (CNTF), glial cell line-derived neurotrophic factor (GDNF), brain-derived neurotrophic factor (BDNF) and mixtures thereof; and the cell nutritional factor is selected from a group comprising fibroblast growth factor (FGF), insulin-like growth factor (IGF), or transforming growth factor (TGF) and mixtures thereof. In another embodiment, the extracellular matrix is NT-3/fibroin complex. In another embodiment, besides the extracellular matrix, the scaffold further comprises a cell secreting extracellular matrix, wherein the cell secreting extracellular matrix is selected from a group comprising Schwann cell, mesenchymal stem cell and fibroblast.

2. Seeding of Neural Stem Cells (NSCs)

2.1 Isolation and Cultivation of NSCs In Vitro

Brains of two one-day-old Sprague Dawley (SD) neonatal rats were taken out under sterile conditions, and were placed in cold D-Hank's solution. The hippocampus was isolated from the brains using an anatomical microscope, and cut into pieces by ophthalmic scissors, transferred into a centrifuge tube with D-Hank's solution, gently pipetted with a fine glass pipette several times until tissue fragments were not visible (the pipetting should be slow and gentle to avoid making bubbles), and centrifuged under 1000 rpm for 5 min to discarded the supernatant. The precipitations of NSCs after the centrifugation were suspended again with D-Hank's solution, said pipetting and centrifugation procedures were repeated. The obtained precipitations of NSCs were suspended with NSCs basal medium, then gently pipetted, diluted to control the cell density to about 1×10⁵ cells/mL. The cell suspension was transferred to a cultivation flask and incubated in an incubator under 37° C. and 5% CO₂. Half of the liquid volume of the NSCs culture medium was renewed and gently pipetted every two days.

2.2 Seeding of NSCs

NSCs that had been cultured for 5 days were seeded into the extracellular matrix-modified decellularized optic nerve scaffolds, Neurobasal medium were added. The NSCs-seeded extracellular matrix-modified decellularized optic nerve scaffolds were cultured for 7 days, during which the Neurobasal medium were renewed every two days.

3. Seeding of Dorsal Root Ganglia (DRG)

3.1 Isolation of DRG In Vitro

Spinal cords of one-day-old green fluorescent protein (GFP) transgenic neonatal rats were taken out under a dissecting microscope and then placed in a culture dish containing pre-cooled DMEM medium. All operations were done on an ice box. DRGs were cut off using a microscissors and were placed in a culture dish containing 2 mL of pre-cooled DMEM medium.

3.2 Seeding of Dorsal Root Ganglia (DRG)

PLGA tubes each with a diameter of 3 mm and a height of 3 mm were placed on a 96-well plate which was coated with Matrigel for stabilizing the PLGA tubes. The prepared extracellular matrix-modified decellularized optic nerve scaffolds were inserted into the PLGA tubes with the transversal cross-sectional surfaces upwardly arranged, gently added with DRGs on the transversal cross-sectional surfaces, added with Neurobasal medium, incubated in an incubator under 37° C. and 5% CO₂ for 7 days, during incubation the Neurobasal medium were renewed every two days.

4. Detection of Cell Growth in Extracellular Matrix-Modified Decellularized Optic Nerve Scaffolds

The extracellular matrix-modified decellularized optic nerve scaffolds seeded with either of the two types of cells, i.e., NSCs and DRG cells, which had been cultured for 7 days, were transversely and longitudinally sectioned with a thickness of 20 μm using a frozen microtome. The cell growths were observed under a fluorescence microscope.

Experiment Results

1. Scaffolds Constructed from Extracellular Matrix and Decellularized Porcine Optic Nerves had a Plurality of Longitudinal Channels and a Plurality of Transversal Foramina.

FIG. 1 is a schematic view of the extracellular matrix-modified decellularized porcine optic nerve scaffold seeded with cells. The scaffold has a plurality of longitudinal channels and a plurality of transversal foramina intercommunicated with the longitudinal channels. On the one hand, the longitudinal channels are beneficial to the extension of synapses of the implanted neurons (seeded cells), the synapses may therefore reach and connect the neurons of host spinal cord through the longitudinal channels. On the other hand, the transversal foramina are beneficial to the communication of the implanted neurons (seeded cells) positioned in different longitudinal channels. Reference numerals and symbols: a: decellularized porcine optic nerve scaffold; 1: longitudinal channel formed in decellularized porcine optic nerve scaffold; 2: neuron; 2-1: neuronal somata; 2-2: nerve fibers.

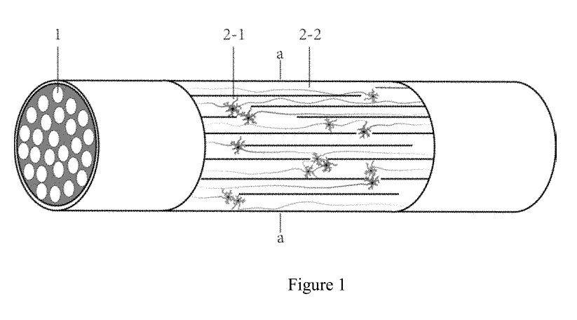

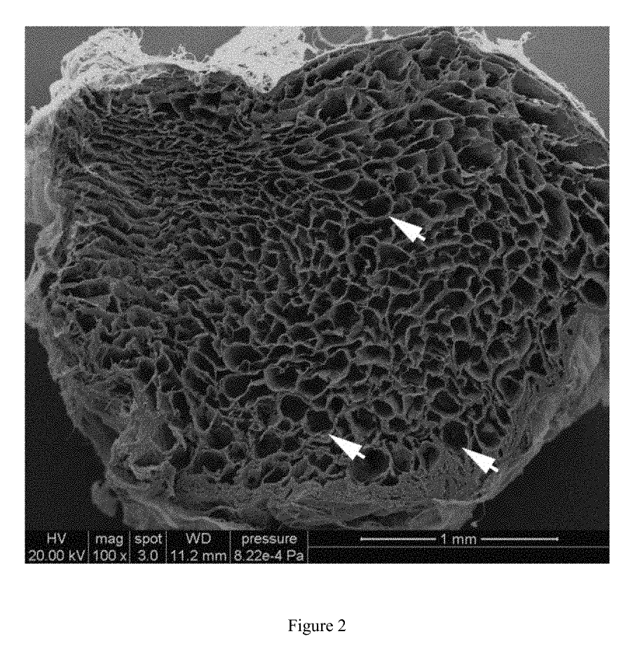

FIG. 2 shows a plurality of longitudinal channels that are divided by cerebral leptomeninx.

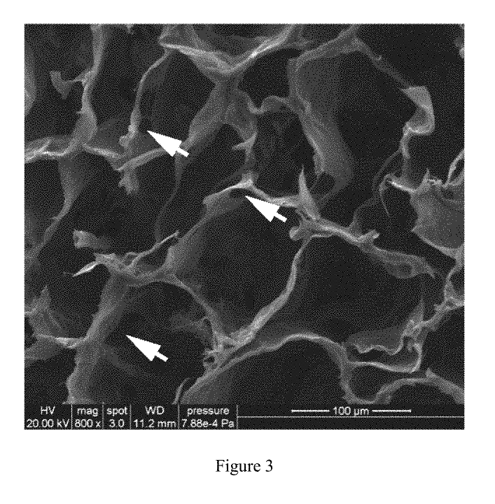

FIG. 3 shows a plurality of transversal foramina intercommunicated with the longitudinal channels.

2. Scaffolds Constructed from Extracellular Matrix and Decellularized Porcine Optic Nerves had a Plurality of Partitions

Referring to FIG. 1, the transversal cross-sectional area of the extracellular matrix-modified decellularized porcine optic nerve scaffold showed a plurality of longitudinal channels each having a circular transversal cross-sectional area. Transversal foramina connected neighbouring longitudinal channels. In this way, the seeded cells may be regionally planted.

3. Three-Dimensional Cylindrical Shape of the Optic Nerve and Longitudinal Channels were Retained in the Decellularized Porcine Optic Nerve Scaffolds

HE staining of natural porcine optic nerves and the constructed extracellular matrix-modified decellularized optic nerve scaffolds showed that the transversal cross-sectional area of the natural porcine optic nerve had a plurality of circular channels filled with nerve fibers depicted by hematoxylin-stained neurons nucleus and eosin-stained cytoplasm and extracellular matrix, the longitudinal cross-sectional area of the natural porcine optic nerve had a plurality of parallel longitudinally-sectioned channels. After the removal of nerve fibers (neurons), epineurium around a whole optic nerve retained and effectively maintained the three-dimensional cylindrical shape of the decellularized porcine optic nerve scaffolds, cerebral leptomeninx extended into the interior of the optic nerve also retained and divided the interior of the optic nerve to form a plurality of channels intercommunicated with transversal foramina.

4. Assessment of Residual DNA

Hoechst33342 staining showed that natural porcine optic nerves contained a large number of blue-colored nuclei under the fluorescence microscope, while no blue-colored nuclei were seen in decellularized porcine optic nerve scaffolds, indicating that the resulting scaffolds are sufficiently decellularized to obviate adverse host immune responses.

4. Cytocompatibility

DRG cells with GFP gene were implanted and cultured for 7 days in the extracellular matrix-modified decellularized porcine optic nerve scaffold. Green DRG cells were seen attached to the surface of the scaffold under fluorescence microscopy. Green nerve fibers (cell protrusions) migrated inward along the longitudinal channels of the scaffold, and the nerve fibers contacted each other through the transversal foramina to form a network. The structure of the longitudinal channels and transversal foramina was conducive to long-distance cell growth and information exchange.

NSCs with GFP gene were implanted and cultured for 7 days in the extracellular matrix-modified decellularized porcine optic nerve scaffold. NSCs shown under fluorescence microscopy were distributed evenly in the longitudinal channels whose walls are formed by cerebral leptomeninx and were connected through the transversal foramina. The cerebral leptomeninx were conducive to cell implantation compartmentally, providing a good structural basis for implantation of different cells in different regions of the scaffold.

The above-mentioned embodiments are the preferred embodiments of the present invention. Variations and modifications are allowed within the scope of the invention. Those skilled in the art will appreciate that the features described above can be combined in various ways to form multiple variations of the invention. As a result, such variations fall within the scope of the protection to the present invention.

What is claimed is:

1. An extracellular matrix-modified decellularized nerve scaffold, characterized in that the scaffold is derived from a natural porcine optic nerve, the scaffold has a plurality of longitudinal channels and a plurality of transversal foramina intercommunicated with the longitudinal channels, and the scaffold is loaded with extracellular matrix.

2. The extracellular matrix-modified decellularized nerve scaffold of claim 1, characterized in that the scaffold is prepared by decellularization, the plurality of longitudinal channels and the plurality of transversal foramina have relatively uniform diameters and relatively even distributions in the scaffold.

3. The extracellular matrix-modified decellularized nerve scaffold of claim 1, characterized in that the extracellular matrix is a cell growth factor and/or a cell nutritional factor;
wherein the cell growth factor is selected from a group comprising neurotrophic factor-3 (NT-3), ciliary neurotrophic factor (CNTF), glial cell line-derived neurotrophic factor (GDNF), brain-derived neurotrophic factor (BDNF) and mixtures thereof; and
the cell nutritional factor is selected from a group comprising fibroblast growth factor (FGF), insulin-like growth factor (IGF), or transforming growth factor (TGF) and mixtures thereof.

4. The extracellular matrix-modified decellularized nerve scaffold of claim 3, characterized in that the scaffold further comprises a cell secreting extracellular matrix, wherein the cell secreting extracellular matrix is selected from a group comprising Schwann cell and mesenchymal stem cell.

5. The extracellular matrix-modified decellularized nerve scaffold of claim 1, characterized in that the scaffold is seeded with seed cells to form a neural network-like structure which is able to be transplanted to an injured spinal cord and thus repair the injured spinal cord.
